(19) United States
(12) Patent Application Publication
(10) Pub. No.: US 2003/0129164 A1
(43) Pub. Date: Jul. 10, 2003

(54) Expression of glial-derived neurotrophic factor for treatment of diseases of the eye

(76) Inventors: John G. Flannery, Berkeley, CA (US); William W. Hauswirth, Gainesville, FL (US)

Correspondence Address: BOZICEVIC, FIELD & FRANCIS LLP, 200 MIDDLEFIELD RD, SUITE 200, MENLO PARK, CA, 94025, US

(21) Appl. No.: 10/308,875

(22) Filed: Dec. 2, 2002

Related U.S. Application Data
(60) Provisional application No. 60/336,889, filed on Dec. 3, 2001.

(51) Int. Cl. A61K 48/00; C12N 15/861

(52) U.S. Cl. 424/93.2; 435/456

(57) ABSTRACT

The invention features methods and compositions for the treatment of disease of the eye, such as retinitis pigmentosa (RP) and glaucoma, by delivery of a neurotrophic factor, particularly glial cell-derived neurotrophic factor (GDNF) using a gene delivery vector. In one embodiment, the gene delivery vector is recombinant viral vector, particularly a recombinant adeno-associated viral (rAAV) vector.

### CROSS-REFERENCE TO RELATED APPLICATION

This application claims the priority benefit of U.S. provisional application serial No. 60/336,889, filed Dec. 3, 2001, which application is incorporated herein by reference in its entirety.

### GOVERNMENT RIGHTS

This invention was made with government support under grant no T32 EY07043 from the National Institutes of Health. The United States government may have certain rights in this invention.

### FIELD OF THE INVENTION

The invention generally relates to compositions and methods for the treatment of diseases of the eye, particularly to the use of gene delivery vectors which direct expression of a neurotrophic factor for treatment of diseases of the eye.

### BACKGROUND OF THE INVENTION

Eye diseases represent a significant health problem in the U.S. and around the world. A wide variety of eye diseases can cause visual impairment, including for example, macular degeneration, diabetic retinopathies, inherited retinal degeneration such as retinitis pigmentosa, glaucoma, retinal detachment or injury and retinopathies (whether inherited, induced by surgery, trauma, a toxic compound or agent, or, photically).

The retina can be particularly affected by in eye disease. The retina, a structure located at the back of the eye, is a specialized light-sensitive tissue that contains photoreceptor cells (rods and cones) and neurons connected to a neural network for the processing of visual information. This information is sent to the brain for decoding into a visual image.

The retina depends on cells of the adjacent retinal pigment epithelium (RPE) for support of its metabolic functions. Photoreceptors in the retina, perhaps because of their huge energy requirements and highly differentiated state, are sensitive to a variety of genetic and environmental insults. The retina is thus susceptible to a variety of diseases that result in visual loss or complete blindness.

An example of such a disease is the blinding disease Retinitis Pigmentosa (RP), which is a candidate for a neuroprotective treatment strategy with techniques of gene therapy. RP is a heterogeneous group of inherited disorders, each characterized by the degeneration of rods, cones, and the RPE in the human retina. The degenerative process and photoreceptor neuronal cell death generally takes place over the course of many years. Mutations which cause RP have been identified in many of the rod and cone photoreceptor genes involved in the phototransduction cascade, including those for rhodopsin, alpha- and beta-subunits of rod cGMP-phosphodiesterase, alpha-subunit of the rod cGMP-gated channel, arrestin, and RP GTPase regulator (Phelan, et al. (2000) Mol. Vis. 6: (2), 116-124). Other RP causing mutations have been detected in genes that code for proteins involved in photoreceptor and RPE structure and metabolism, including RDS, ROM1, cellular retinaldehyde binding protein, RPE65, myosin VIIA, and ABCA4 (Phelan, et al. (2000) Mol. Vis. 6: (2), 116-124). Rhodopsin mutations are most prevalent and account for approximately 10 percent of all cases. Many diseases are monogenic, generated by one mutation in one gene, but this heterogeneous group of diseases which are collectively called RP is unusual in that so many different mutations produce a similar disease phenotype. For RP therefore, it may be important to assess the utility of non-gene specific forms of therapy that could be employed against a variety of RP disease types.

Other diseases of the eye, such as glaucoma, are also major public health problems in the United States. Glaucoma is not a uniform disease but rather a heterogeneous group of disorders that share a distinct type of optic nerve damage that leads to loss of visual function. The disease is manifest as a progressive optic neuropathy that, if left untreated, leads to blindness. Glaucoma can involve several tissues in the front and back of the eye. Commonly, but not always, glaucoma begins with a defect in the front of the eye. Fluid in the anterior portion of the eye, the aqueous humor, forms a circulatory system that brings nutrients and supplies to various tissues. Aqueous humor enters the anterior chamber via the ciliary body epithelium (inflow), flows through the anterior segment bathing the lens, iris, and cornea, and then leaves the eye via specialized tissues known as the trabecular meshwork and Schlemm's canal to flow into the venous system. Intraocular pressure is maintained vis-α-vis a balance between fluid secretion and fluid outflow. Almost all glaucomas are associated with defects that interfere with aqueous humor outflow and, hence, lead to a rise in intraocular pressure. The consequence of this impairment in outflow and elevation in intraocular pressure is that optic nerve function is compromised. The result is a distinctive optic nerve atrophy, which clinically is characterized by excavation and cupping of the optic nerve, indicative of loss of optic nerve axons.

Primary open-angle glaucoma, the most prevalent form of glaucoma, is, by convention, characterized by relatively high intraocular pressures believed to arise from a blockage of the outflow drainage channel or trabecular meshwork in the front of the eye. However, another form of primary open-angle glaucoma, normal-tension glaucoma, is characterized by a severe optic neuropathy in the absence of abnormally high intraocular pressure. Patients with normal-tension glaucoma have pressures within the normal range, albeit often in the high normal range. Both these forms of primary open-angle glaucoma are considered to be late-onset diseases in that, clinically, the disease first presents itself around midlife or later. However, among African-Americans, the disease may begin earlier than middle age. In contrast, juvenile open-angle glaucoma is a primary glaucoma that affects children and young adults. Clinically, this rare form of glaucoma is distinguished from primary open-angle glaucoma not only by its earlier onset but also by the very high intraocular pressure associated with this disease.

Primary open-angle glaucoma can be insidious. It usually begins in midlife and progresses slowly but relentlessly. If detected, disease progression can frequently be arrested or slowed with medical and surgical treatment. However, without treatment, the disease can result in absolute irreversible blindness. In many cases, even when patients have received adequate treatment (e.g., drugs to lower intraocular pressure), optic nerve degeneration and loss of vision continues relentlessly.

Angle-closure glaucoma is a mechanical form of the disease caused by contact of the iris with the trabecular meshwork, resulting in blockage of the drainage channels that allow fluid to escape from the eye. This form of glaucoma can be treated effectively in the very early stages with laser surgery. Congenital and other developmental glaucomas in children tend to be severe and can be very challenging to treat successfully. Secondary glaucomas result from other ocular diseases that impair the outflow of aqueous humor from the eye and include pigmentary glaucoma, pseudoexfoliative glaucoma, and glaucomas resulting from trauma and inflammatory diseases. Blockage of the outflow channels by new blood vessels (neovascular glaucoma) can occur in people with retinal vascular disease, particularly diabetic retinopathy.

Neurotrophic factors are known to modulate neuronal growth during development to maintain existing cells and to allow recovery of injured neuronal populations. Observations of retinal neurons during development (Crespo et al., (1985) Brain Research 351: (1), 129-134) suggest that correct synaptic connections are reinforced by trophic factors, while cells that make inappropriate connections and do not receive trophic support undergo apoptosis. Hence, it has long been hypothesized that if the removal of neurotrophic factors from the cellular environment can stimulate cell death then adding exogenous trophic factors may have neuroprotective effects in the retina (Faktorovich, et al. (1990) Nature 347: (6288), 83-86).

GDNF was first described as a stimulant of survival of dopaminergic neurons in-vitro (Lin, et al. (1993) Science 260: (5111), 1130-1132) and was found to belong to the transforming growth factor-beta superfamily. Shortly after its discovery, it was demonstrated to have protective effects in in-vivo models of Parkinson's Disease (Kaddis, et al. (1996) Cell Tissue Res. 286: (2), 241-247; Gash, et al. (1996) Nature 380: (6571), 252-255; Choi-Lundberg, et al. (1997) Science 275: (5301), 838-841), on dorsal root ganglion neurons (Matheson, et al. (1997) J. Neurobiol. 32: (1), 22-32), and on motor neurons during development (Oppenheim, et al. (1995) Nature 373: (6512), 344-346). GDNF interacts with a specific cell-surface receptor, GFRA1 (Jing, et al. (1996) Cell 85: (7), 1113-1124; Treanor, et al. (1996) Nature 382: (6586), 80-83), and its biological effects are mediated through the interaction of GDNF, GFRA1, and a tyrosine kinase receptor, RET (Takahashi, et al. (1987) Mol Cell Biol 7: (4), 1378-1385). Both GDNF and its receptors are synthesized in the retina (Jing, et al. (1996) Cell 85: (7), 1113-1124; Nosrat, et al. (1996) Cell Tissue Res. 286: (2), 191-207; Pachnis, et al. (1993) Development 119: (4), 1005-1017). GDNF protein have been examined in photoreceptors in the Pde6b^{-/-} (rd) mouse (Frasson, et al. (1999) Invest. Ophthalmol. Vis. Sci. 40: (11), 2724-2734), in photoreceptor outer segment collapse in-vitro (Carwile, et al. (1998) Exp. Eye Res. 66: (6), 791-805), and in mouse photoreceptors in-vitro (Jing, et al. (1996) Cell 85: (7), 1113-1124).

A great deal of the progress made in addressing the important clinical problems of conditions such as RP and glaucoma has depended on advances in research on photoreceptor cell biology, molecular biology, molecular genetics, and biochemistry over the past two decades. Animal models of hereditary retinal disease have been vital in helping unravel the specific genetic and biochemical defects that underlie abnormalities in human retinal diseases. It now seems clear that both genetic and clinical heterogeneity underlie many hereditary retinal diseases.

A number of neurotrophins have been tested for their ability to support photoreceptor survival in various models of retinal degeneration (Frasson, et al. (1999) Invest. Ophthalmol. Vis. Sci. 40: (11), 2724-2734; Cayouette, et al. (1997) Hum. Gene. Ther. 8: (4), 423-430; LaVail, et al. (1998) Invest. Ophthalmol. Vis. Sci. 39: (3), 592-602; Lau, et al. (2000) Invest. Ophthalmol. Vis. Sci. 41: (11), 3622-3633; Jablonski, et al. (2000) J. Neuroscience 20: (19), 7149-7157). Photoreceptors have high oxygen and nutrient demands and must maintain a complex equilibrium of extracellular and intracellular ions for phototransduction. This makes rods and cones particularly susceptible to genetic, structural, and biochemical insults (Travis (1998) Am. J. Hum. Genet. 62: (3), 503-508; Stone, et al. (1999) Prog. Retin. Eye. Res. 18: (6), 689-735).

Disturbances in the visual cycle appear to trigger apoptotic cell death in photoreceptors.

Substantial effort in retinal degeneration research has focused on the therapeutic effect of neurotrophins as a general protective strategy to slow the progression of degeneration. Specific gene therapies, such as antisense or ribozymes (Lewin, et al. (1998) Nat. Med. 4: (8), 967-971), which work to eliminate mutant mRNA of the affected gene, have promise for treating dominant forms of RP. Unfortunately, different ribozyme or antisense therapies must be designed for each specific mutation. Gene replacement may be used as a therapy for recessive forms of RP (Lem, et al. (1992) Proc Natl Acad Sci USA 89: (10), 4422-4426; Travis, et al. (1992) Neuron 9: (1), 113-119; Bennett, et al. (1996) Nat. Med. 2: (6), 649-654), but it cannot readily treat the majority of RP patients. An alternative to these gene-specific therapies is generalized survival factor therapy that does not target the mutant gene product, but alters the photoreceptor environment in a manner promoting cell survival. The aim is to slow the rate of cell death therefore prolonging the period of useful vision for patients.

LaVail, Steinberg, and colleagues pioneered this field by testing many different survival factors in rat models of photoreceptor degeneration (Faktorovich, et al. (1990) Nature 347: (6288), 83-86; Faktorovich, et al. (1992) J. Neurosci. 12: (9), 3554-3567; LaVail, et al. (1992) Proc Natl. Acad. Sci. USA 89: (23), 11249-11253; see also U.S. Pat. No. 5,667,968). They noted a slowing of photoreceptor cell death with direct protein injections of different growth factors or neurotrophic agents, including basic fibroblast growth factor (FGF2), CNTF, and BDNF. However, prolonged rescue of photoreceptor degeneration by intraocular injection of protein has been difficult to achieve because therapeutic proteins are continuously degraded in the body and lose biological activity over a short period of time. Theoretically, the rescue seen with protein injections could be sustained with repetitive delivery; however, repetitive injection of survival factors into the subretinal space is not a practical regimen for RP patients.

Gene delivery methods hold promise because photoreceptor cells, if properly transduced, can continually produce their own neurotrophic factor. One vector of interest for retinal gene therapy in humans is recombinant adeno-associated virus (rAAV) (Hauswirth, et al. (2000) Invest Ophthalmol Visual Sci 41: (10), 2821-2826; see also WO 00/54813). When injected subretinally, rAAV delivers the gene of interest to photoreceptors and to the RPE (Acland, et al. (2001) Nature Genetics 28: (1), 92-95). Additionally, recombinant AAV vectors are not associated with any known human disease. Moreover, recent improvements in rAAV production have made manufacturing of high titer gene transfer vector easily attainable. In a previous study using AAV to transduce the retina, the expression levels increased progressively after 1 week post-injection and plateau at approximately 5 weeks post-injection (McGee Sanftner, et al. (2001) Mol. Ther. 3: (5 Pt 1), 688-696).

Despite advances in the field, the optimal neurotrophic factor for delivery to the retina and treatment eye diseases has not yet been identified in the art. For example, while the neurotrophic growth factors (e.g., fibroblast growth factors), appear promising (see, e.g., WO 00/54813), there are concerns that such factors may also promote new blood vessel formation, placing a patient at risk of, for example, a macular degenerative-type disorder, particularly in individuals who are susceptible macular degeneration. Furthermore, while some therapies rescue the cells from cell death, preserving the physiology of the cell, little success has been reported to date in the protection of cells in a manner that preserves the electrophysiologic response of the retina to light. The present invention solves these problems.

### SUMMARY OF THE INVENTION

The invention features methods and compositions for the treatment of disease of the eye, such as retinitis pigmentosa (RP) and glaucoma, by delivery of a neurotrophic factor, particularly glial cell-derived neurotrophic factor (GDNF) using a gene delivery vector. In one embodiment, the gene delivery vector is recombinant viral vector, particularly a recombinant adeno-associated viral (rAAV) vector.

In one aspect the invention features a method of reducing the degeneration of photoreceptors in a subject having or susceptible to an eye disease or condition (e.g., a disease or condition that results from exposure to an environmental condition or to the presence of a pathological condition) which causes or places the subject at risk of such photoreceptor degeneration, the method comprising administering to the patient a recombinant gene delivery vector adapted for expression of GDNF, wherein GDNF is expressed in the subject's eye in an amount sufficient to treat the condition.

In another aspect, the invention features a method of promoting the regeneration of photoreceptors in a subject having an eye condition or disease, where the method comprises administering into the patient's eye a gene delivery vector for expression of an effective amount of GDNF.

In specific embodiments, the recombinant gene delivery vector is a recombinant viral vector, with a recombinant AAV vector being particularly preferred.

In specific embodiments, the gene delivery vector is administered by intraocular administration, e.g., into the vitreous or into the interphotoreceptor space.

In further specific embodiments, the eye condition is an environmental or pathological condition selected from the group consisting of retinitis pigmentosa (RP), glaucoma, retinal detachment, age-related or other maculopathies, photic retinopathies, surgery-induced retinopathies, toxic retinopathies, retinopathy of prematurity, retinopathies due to trauma or penetrating lesions of the eye, or inherited retinal degenerations.

One advantage of the invention is that expression of GDNF in the eye facilitates preservation of photoreceptor structure and function, and in a manner that is significantly better than other neurotrophic factors (NTFs). Other NTFs (specifically, CNTF and axokine) have been shown to suppress the electrophysiologic response of the retina to light.

Another advantage of the invention is that the eye condition or disease can be treated without the risk of inducing neovascularization. In contrast, NTFs such as fibroblast growth factors (FGFs), pose the risk of this potential negative side effect of neovascularization.

Another advantage of the invention is that the expression of GDNF in the eye takes advantage of the presence of GDNF receptors on the cell of interest for protection, i.e., photoreceptors themselves (as compared to elsewhere in the retina). In contrast, the receptors for many other NTFs are localized to Muller glia in the retina, and not on the photoreceptors themselves. For example, the TRKa, TRKb and TRKc receptors for the FGFs, CNTF, BDNF, and several NTFs are not found on the photoreceptors, but on other retinal cell types unaffected by the disease. Without being held to theory, other NTFs thus likely work indirectly on photoreceptor survival through an paracrine pathway, in which those NTFs bind to glia, and the glia in response to this secrete a factor or factors which protect(s) the photoreceptors from injury. In contrast, since the GDNF receptors are on the photoreceptors themselves, the resulting protective effect is directly acting upon the cells which have the genetic defect and are dying from the disease, providing a more immediate, local effect at the desired site of treatment.

These and other advantages, and features of the invention will become apparent to those persons skilled in the art upon reading the details of the invention as more fully described below.

### BRIEF DESCRIPTION OF THE DRAWINGS

FIG. 1 is a photograph of a Western blot for GDNF. Western blot analysis of total cell lysate from AAV transfected retinas probed for human recombinant GDNF. Kaleidoscope pre-stained protein standard marker (lane 1). Retinal protein from five weeks post AAV-CBA-GDNF injection (lanes 2, 3, and 4). Retinal protein from an uninjected rat (lane 5). Retinal protein five weeks post AAV-CBA-GFP injection (lane 6). Human recombinant GDNF protein control (lane 7). Expression of human recombinant GDNF protein is specific to AAV-CBA-GDNF-injected retinas (lanes 2, 3, and 4), and absent from uninjected (lane 5), or AAV-CBA-GFP-injected retinas (lane 6).

FIG. 2 is panel of photographs of GDNF immunohistochemistry. In a cross-section through the injection site of an AAV-CBA-GDNF-treated retina, immunostaining for human recombinant GDNF is localized to photoreceptor nuclei (Panel A) and RPE cells (Panel C). Human recombinant GDNF staining was absent from untreated retina (Panel B) and RPE cells (Panel D). ONL, outer nuclear layer; INL, inner nuclear layer; GCL, ganglion cell layer; CHR, choroid; RPE, retinal pigment epithelium. Scale bar=25 μm.

FIG. 3 is a photograph showing the results of RT-PCR detection of GDNF expression. Expression of human recombinant GDNF mRNA transcripts is specific to AAV-CBA-GDNF-treated retinas and absent from untreated retinas. Primers that amplify both rat and human GDNF (lanes 1, 3, and 5), or AAV-CBA-GDNF-derived human GDNF (lanes 2, 4, and 6) were used. CBA-GDNF plasmid DNA was used as a positive control and supports amplification of only human GDNF, 641 bp and 511 bp bands. Retinal cDNA from untreated animals supports amplification of only endogenous rat GDNF, 641 bp band. Retinal cDNA from AAV-CBA-GDNF-treated animals supports amplification of both endogenous rat GDNF and AAV-CBA-GDNF-derived human GDNF, 641 bp and 511 bp bands. CBA-GDNF plasmid DNA, (lanes 1 and 2). Retinal cDNA for an untreated rat, (lanes 3 and 4). Retinal cDNA five weeks post AAV-CBA-GDNF treatment, (lanes 5 and 6). DNA ladder, (lane 7). PCR products were amplified from cDNA of individual eyes and representative samples from each group are shown.

FIG. 4 is a graph showing mean ONL thickness measurements in the superior region of AAV-CBA-GDNF-injected, AAV-CBA-GFP-injected, and uninjected retinas of TgN S334ter-4 rats at P60. AAV-CBA-GDNF-treated eyes had an increase in mean ONL thickness, 23.8±4.1 μm compared to 16.3±2.5 μm in AAV-CBA-GFP-treated or 15.4±2.2 μm in uninjected affected controls. Mann-Whitney test analysis indicated that the mean ONL thickness in the superior region of AAV-CBA-GDNF-injected retinas was significantly increased in comparison to both controls (P<0.0002). AAV-CBA-GFP-injected and uninjected affected controls were not statistically significantly different from one another (P>0.05). n=number of eyes. n=24 for AAV-CBA-GDNF-injected, n=9 for AAV-CBA-GFP-injected, and n=9 for uninjected affected controls. Error bars show the standard deviation among averaged ONL thickness.

FIG. 5 is a graph showing mean ONL thickness measurements in the inferior region of AAV-CBA-GDNF-injected, AAV-CBA-GFP-injected, and uninjected retinas of TgN S334ter-4 rats at P60. AAV-CBA-GDNF-treated eyes had an increase in mean ONL thickness, 28.8±2.8 μm, compared to 21.9±3.0 μm in AAV-CBA-GFP-treated, or 21.4±2.6 μm in uninjected affected controls. Mann-Whitney test analysis indicated that the mean ONL thickness in the inferior region of AAV-CBA-GDNF-injected retinas was significantly increased in comparison to both controls (P<0.0002). AAV-CBA-GFP-injected and uninjected affected controls were not statistically significantly different from one another (P>0.05). n=number of eyes. n=24 for AAV-CBA-GDNF-injected, n=9 for AAV-CBA-GFP-injected, and n=9 for uninjected affected controls. Error bars show the standard deviation among averaged ONL thickness.

FIG. 6 is a series of photographs showing morphological rescue of TgN S334ter-4 superior retinas at P60. In the superior hemisphere of uninjected TgN S334ter-4 retinas, photoreceptors degenerate to 2 to 3 cells thick. Retinas injected with AAV-CBA-GDNF had an ONL that is significantly thicker than AAV-CBA-GFP-injected or uninjected affected controls. Inner and outer segments of photoreceptors are also more organized and better preserved in AAV-CBA-GDNF-treated animals than in AAV-CBA-GFP-treated or untreated affected controls. RPE, retinal pigment epithelium; ONL, outer nuclear layer; INL, inner nuclear layer; GCL, ganglion cell layer. Scale bar=20 μm.

FIG. 7 is a series of representative electroretinographic intensity-response functions from TgN S334ter-4 eyes at P60, which were AAV-CBA-GDNF-treated (Panel A), untreated (Panel B), or AAV-CBA-GFP-treated (Panel C). The electroretinograms were recorded in dark-adapted conditions using flashes of white light, the intensity of which is indicated in log cd-s/m² at the left of each trace. The vertical arrows point to flash onset. The amplitude of both the a- and b-wave of affected eyes having received a subretinal injection of AAV-CBA-GDNF (Panel A), are noticeably larger compared with that of untreated affected animals (Panel B), or AAV-CBA-GFP-treated animals (Panel C).

FIG. 8 is a table showing the mean a- and b-wave amplitudes from ERG analysis of TgN S33ter-4 mutants.

### DETAILED DESCRIPTION OF PREFERRED EMBODIMENTS

Before the present invention is described, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used in the specification in its entirety, the singular forms “a”, “and”, and “the” include plural referents unless the context clearly dictates otherwise. Thus, for example, reference to “a cell” includes a plurality of such cells and reference to “the vector” includes reference to one or more vectors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

“Gene” as used herein is meant to refer to at least a polynucleotide having at least a minimal sequence required for the expression of a coding sequence of interest. For example, “gene” minimally comprises a promoter that, when operably linked to a coding sequence of interest, facilitates expression of the coding sequence in a host cell. The coding sequence of the “gene” can be a genomic sequence (which includes one or more introns and exons) which, following splicing or rearrangement, provide for expression of a gene product of interest, or a recombinant polynucleotide, which lacks some or all intronic sequences (e.g., a cDNA).

The terms “polynucleotide” and “nucleic acid”, used interchangeably herein, refer to a polymeric forms of nucleotides of any length, either ribonucleotides or deoxynucleotides. Thus, these terms include, but are not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. These comprise intronic and exonic sequences. In general, polynucleotides of interest in the present invention are those that are adapted for expression in a eukaryotic host cell, particularly a mammalian host cell, preferably a human cell, especially a cell of the eye (e.g., a retinal cell), particularly a mammalian (preferably human) cell of the eye.

The terms “polypeptide” and “protein”, used interchangeably herein, refer to a polymeric form of amino acids of any length, which in the context of the present invention, generally include amino acid residues that are genetically encodable. Polypeptides can also include those that are biochemically modified (e.g., post-translational modification such as glycosylation), as well as fusion proteins, including, but not limited to, fusion proteins with a heterologous amino acid sequence, fusions with heterologous and homologous leader sequences, with or without N-terminal methionine residues; immunologically tagged proteins; and the like.

The term “recombinant polynucleotide” as used herein intends a polynucleotide of genomic, cDNA, semisynthetic, or synthetic origin which, by virtue of its origin or manipulation: (1) is not associated with all or a portion of a polynucleotide with which it is associated in nature, (2) is linked to a polynucleotide other than that to which it is linked in nature, or (3) does not occur in nature.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

An “open reading frame” (ORF) is a region of a polynucleotide sequence that encodes a polypeptide; this region may represent a portion of a coding sequence or a total coding sequence.

A “coding sequence” is a polynucleotide sequence that is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, and recombinant polynucleotide sequences.

“Transformation”, as used herein, refers to the insertion of an exogenous polynucleotide into a host cell, irrespective of the method used for the insertion, for example, viral infection, direct uptake, transduction, f-mating or electroporation. The exogenous polynucleotide may be maintained as a non-integrated vector, for example, an episomal element, or alternatively, may be integrated into the host genome.

“Subjects” or “patients” as used herein is meant to encompass any subject or patient amenable to application of the methods of the invention. Subjects include, without limitation, primate, canine, feline, bovine, equine, ovine, and avian subjects; mammals (particularly humans), domesticated pets (e.g., cat, dogs, birds, etc.) and livestock (cattle, swine, horses, etc.), and zoo animals being of particular interest.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Gene delivery vector” refers to a construct that is adapted for delivery of, and, within preferred embodiments facilitating expression, one or more gene(s) or sequence(s) of interest in a host cell. Representative examples of such vectors include viral vectors, nucleic acid expression vectors, naked DNA, and certain eukaryotic cells (e.g., producer cells).

“Recombinant adeno-associated virus vector” or “rAAV vector” refers to a gene delivery vector based upon an adeno-associated virus. The rAAV vectors, generally contain 5′ and 3′ adeno-associated virus inverted terminal repeats (ITRs), and a transgene or gene of interest operatively linked to sequences which regulate its expression in a target cell. Within certain embodiments, the transgene may be operably linked to a heterologous promoter (such as a CMV, with the CBA promoter described herein being of particular interest), or, an inducible promoter such as (tet). In addition, the rAAV vector may have a polyadenylation sequence.

“Diseases of the eye” or “eye condition” refers to a broad class of diseases or conditions wherein the functioning of the eye is affected due to damage or degeneration of the photoreceptors; or ganglia or optic nerve. Representative examples of such diseases include macular degeneration, diabetic retinopathies, inherited retinal degeneration such as retinitis pigmentosa, glaucoma, retinal detachment or injury and retinopathies (whether inherited, induced by surgery, trauma, a toxic compound or agent, or, photically).

Overview

The present invention is based on the discovery that the neurotrophic factor glial cell line-derived neurotrophic factor (GDNF) is an effective neuroprotective factor for photoreceptors that would otherwise be destined for apoptosis in a genetic model for disease of the eye, specifically retinitis pigmentosa (RP). Gene transfer of GDNF to the retina was achieved via a recombinant adeno-associated virus (rAAV) vector containing the chicken beta-actin promoter/immediate early CMV enhancer (CBA) driving the human GDNF gene. AAV-CBA-GDNF was delivered to the retinas of an animal model of Retinitis Pigmentosa, the TgN S334ter-4 rhodopsin line of transgenic rats.

Immunohistochemical studies localized AAV-CBA-GDNF-derived recombinant protein to cell bodies, inner segments, and outer segments of photoreceptor cells as well as to retinal pigment epithelial (RPE) cells. GDNF vector treatment was found to lead to increased rod photoreceptor survival as indicated by morphometric analysis of outer nuclear layer (ONL) thickness. AAV-CBA-GDNF-treated retinas also demonstrated functional improvement by the significantly increased amplitude of electroretinograms (ERG). AAV-CBA-GDNF delivery had a significant rescue effect on photoreceptor degeneration in this animal model.

Accordingly, the invention provides methods and compositions useful in the treatment of a variety of diseases of the eye that can be treated by delivery of a neurotrophic factor, such as GDNF, for protection of photoreceptors from cell death.

The invention will now be described in more detail.

Glial-Derived Neurotrophic Factor (GDNF)

GDNF is a neurotrophic factor of particular interest for delivery according to the invention. The gene delivery vector suitable for use in the invention can provide for expression of GDNF from any suitable polynucleotide sequence, e.g., a genomic sequence or cDNA. The coding sequence can provide for expression of a full-length GDNF polypeptide (mature or pre-processed (‘immature) forms), or for a biologically active portion thereof (e.g., a polypeptide having amino acid sequence deletions (e.g., truncations), insertions, additions, or substitutions that do not affect the activity of GDNF in neuroprotection as required by the methods described herein).

GDNF is a member of a family of ligands within the TGF-beta superfamily of signaling molecules. Members of the GDNF family of ligands includes GDNF, neurturin (NTN), persephin (PSP), and artemin (ART). GDNF was first characterized by Lin et al. ((1993) “GDNF, a glial cell line-derived neurotrophic factor for midbrain dopaminergic neurons.” Science 260:1130-1132) as a protein secreted from the rat B49 glial cell line. The protein was purified and partially sequenced, and oligomer probes were designed to clone both the human and rat cDNAs. The predicted 211-amino acid sequences of the 2 proteins are 93% identical. The larger product is processed to a mature 134-amino acid secreted form that occurs as a homodimer. The mature GDNF protein contains 7 conserved cysteine residues spaced similarly to those of members of the TGF-beta superfamily of proteins, to which it is weakly related. GDNF has been mapped to the human genome at Gene map locus 5p13.1-p12

All members of the GDNF family exhibit neurotrophic properties, and thus are useful in the present invention. For example, artemin has been shown in culture to support the survival of a number of peripheral neuron populations and at least one population of dopaminergic central nervous system (CNS) neurons. Its role in the peripheral nervous systems (PNS) and CNS is further substantiated by its expression pattern in the proximity of these neurons. This protein is a ligand for the RET receptor and uses GFR-alpha 3 as a coreceptor. Four alternatively spliced transcripts have been described, two of which encode the same protein.

GDNF transcript variants having neurotrophic properties are contemplated for use in the present invention. Several such GDNF transcript variants have been described. For example, the GDNF transcript variant described at GenBank Accession No. NP_{—}476501 has a different 5′ end sequence as compared to variant 1 and encodes an isoform with a unique amino terminus.

The GDNF for delivery according to the method of the invention can be derived from any suitable source. Where the subject is human, the GDNF is preferably derived from a human or human-compatible (e.g., a GDNF that is from an origin other than human, but provides for acceptable biological activity in neuroprotection in human cells) GDNF polypeptide. The GDNF-coding sequence of the gene delivery vector can be produced by any suitable method, and is generally recombinantly produced.

Polynucleotide sequences encoding GDNF that may be of use are those described at, for example, GenBank Accession Nos. L19063, L15306, XM_{—}031129; and at Lin et al. (1993) GDNF, a glial cell line-derived neurotrophic factor for midbrain dopaminergic neurons. Science 260:1130-1132.

GDNF polypeptides and sequences encoding such polypeptides suitable for use in the invention are further described in, for example, U.S. Pat. Nos. 5,733,875; 5,731,284; 5,736,516; 5,741,778; 6,093,802; and 6,221,376. Exemplary GDNF variants suitable for use in the invention are described in U.S. Pat. No. 6,184,200. “GDNF” as used herein thus is meant to encompass mature and immature forms of the protein, as well as variants (e.g., naturally-occurring or engineered GDNF having one or more amino acid insertions, deletions, additions, or substitutions, including splice variants, and the like).

Gene Delivery Vectors

Any of a variety of vectors adapted for expression of GDNF in a cell of the eye, particularly within a retinal cell, more particularly within a photoreceptor cell, are within the scope of the present invention. Gene delivery vectors can be viral (e.g., derived from or containing sequences of viral DNA or RNA, preferably packaged within a viral particle), or non-viral (e.g., not packaged within a viral particle, including “naked” polynucleotides, nucleic acid associated with a carrier particle such as a liposome or targeting molecule, and the like).

Exemplary gene delivery vectors are described below.

Recombinant Adeno-Associated Virus Vectors (rAAV)

A particularly preferred gene delivery vector is an rAAV vector. A variety of rAAV vectors may be utilized to direct the expression of a neurotrophic factor such as GDNF. Briefly, the rAAV is generally comprised of, in order of 5′ to 3′, a 5′ adeno-associated virus inverted terminal repeat, a coding sequence for the desired gene product (e.g., GDNF) operatively linked to a sequence which regulates its expression in a cell (e.g., a promoter sequence), and a 3′ adeno-associated virus inverted terminal repeat. In addition, the rAAV vector may preferably have a polyadenylation sequence. The polynucleotide of interest for delivery to the cell using a vector is sometimes referred to herein without limitation as the “transgene”.

Generally, rAAV vectors should have one copy of the AAV ITR at each end of the transgene or gene of interest, in order to allow replication, packaging, and efficient integration into cell chromosomes. The ITR consists of nucleotides 1 to 145 at the 5′ end of the AAV DNA genome, and nucleotides 4681 to 4536 (i.e., the same sequence) at the 3′ end of the AAV DNA genome. Preferably, the rAAV vector may also include at least 10 nucleotides following the end of the ITR (i.e., a portion of the “D region”).

Within preferred embodiments of the invention, the transgene sequence will be of about 2 to 5 kb in length (or alternatively, the transgene may additionally contain a “stuffer” or “filler” sequence to bring the total size of the nucleic acid sequence between the two ITRs to between 2 and 5 kb). Alternatively, the transgene may be composed of same heterologous sequence several times (e.g., two nucleic acid molecules which encode FGF-2 separated by a ribosome readthrough, or alternatively, by an Internal Ribosome Entry Site or “IRES”), or several different heterologous sequences (e.g., FGF-2 and FGF-5, separated by a ribosome readthrough or an IRES).

Recombinant AAV vectors of the present invention may be generated from a variety of adeno-associated viruses, including for example, serotypes 1 through 6. For example, ITRs from any AAV serotype are expected to have similar structures and functions with regard to replication, integration, excision and transcriptional mechanisms.

Within certain embodiments of the invention, expression of the transgene may be accomplished by a separate promoter (e.g., a viral or other promoter that facilitates expression of an operably linked sequence in a eukaryotic cell, particularly a mammalian cell). Representative examples of suitable promoters in this regard include a CBA promoter (chicken beta actin), CMV promoter, RSV promoter, SV40 promoter, or MoMLV promoter. Other promoters that may similarly be utilized within the context of the present invention include cell or tissue specific promoters (e.g., a rod, cone, or ganglia derived promoter), or inducible promoters. Representative examples of suitable inducible promoters include tetracycline-response promoters (“Tet”, see, e.g., Gossen and Bujard, Proc. Nail Acad. Sci. USA. 59:5547-5551, 1992; Gossen et al., Science 268, 1766-1769, 1995; Baron et al., Nucl. Acids Res. 25:2723-2729, 1997; Blau and Rossi, Proc. Natl. Acad. Sci. USA. 96:191-199, 1999; Bohl et al., Blood P2:1512-1517, 1998; and Haberman et al., Gene Therapy 5:1604-1611, 1998), the ecdysone system (see e.g., No et al., Proc. Natl. Acad. Sci. USA. 95:3346-3351, 1996), and other regulated promoters or promoter systems (see, e.g., Rivera et al., Nat. Med. 2:1028-1032, 1996;).

The rAAV vector may also contain additional sequences, for example from an adenovirus, which assist in effecting a desired function for the vector. Such sequences include, for example, those which assist in packaging the rAAV vector into virus particles.

Packaging cell lines suitable for producing adeno-associated viral vectors may be readily accomplished given readily available techniques (see e.g., U.S. Pat. No. 5,872,005).

Methods for constructing and packaging rAAV vectors are described in, for example, WO 00/54813.

Recombinant Adenoviral Vectors

In other, less preferred, embodiments, the gene delivery vector is a recombinant adenoviral vector. U.S. Pat. No. 6,245,330 describes recombinant adenoviruses coding for GDNF which can may be suitable for use in the invention.

Use of adenoviral vectors (Ad) may be less preferred for use in some instances relative to use of rAAV vectors as described above. For example, because Ad vectors do not integrate into the host cell genome, as a result, gene expression is short term, typically about 14 days. Thus, use of Ad vectors can require repeated intraocular injections to treat a retinal disease which continues over decades in the average patient. Ad vectors can be generated using commonly available techniques.

Any of the above retroviruses may be readily utilized in order to assemble or construct retroviral gene delivery vectors given the disclosure cause significant inflammatory responses to the Ad viral capsid proteins in the eye as well as in numerous other tissues. Finally, the viral tropism of Ad and AAV in the retina is significantly different. The subset of cells that are transduced by the vector is a receptor mediated event. Ad vectors have been shown to primarily transduce retinal Muller cells and Retinal pigment epithelial cells following injection. AAV vectors are very efficient at transferring their genetic payload to photoreceptors, the cell of interest, when injected into the eye.

Construction of Retroviral Gene Delivery Vectors

The gene delivery vectors of the invention can be a retroviral gene delivery vector adapted to express a selected gene(s) or sequence(s) of interest (e.g., GDNF). Retroviral gene delivery vectors of the present invention may be readily constructed from a wide variety of retroviruses, including for example, B, C, and D type retroviruses as well as spumaviruses and lentiviruses (see RNA Tumor Viruses, Second Edition, Cold Spring Harbor Laboratory, 1985). Such retroviruses may be readily obtained from depositories or collections such as the American Type Culture Collection (“ATCC”; Rockville, Md.), or isolated from known provided herein, and standard recombinant techniques (e.g., Sambrook et al., Molecular Cloning: A Laboratory Manual 2d ed., Cold Spring Harbor Laboratory Press, 1989; Kunkel, PNAS 52:488, 1985).

In addition, within certain embodiments of the invention, portions of the retroviral gene delivery vectors may be derived from different retroviruses. For example, within one embodiment of the invention, retrovirus LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

Within one aspect of the present invention, retroviral vector constructs are provided comprising a 5′ LTR, a tRNA binding site, a packaging signal, one or more heterologous sequences, an origin of second strand DNA synthesis and a 3′ LTR, wherein the vector construct lacks gag/pol or env coding sequences.

Other retroviral gene delivery vectors may likewise be utilized within the context of the present invention, including for example EP 0,415,731; WO 90/07936; WO 91/0285, WO 9403622; WO 9325698; WO 9325234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 55:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Packaging cell lines suitable for use with the above described retroviral vector constructs can be readily prepared according to methods well known in the art, and utilized to create producer cell lines for the production of recombinant vector particles.

Alphavirus Delivery Vectors

Gene delivery vectors suitable for use in the invention can also be based upon al phavirus vectors. For example, the Sindbis virus is the prototype member of the alphavirus genus of the togavirus family. The unsegmented genomic RNA (49S RNA) of Sindbis virus is approximately 11,703 nucleotides in length, contains a 5′ cap and a 3′ poly-adenylated tail, and displays positive polarity. Infectious enveloped Sindbis virus is produced by assembly of the viral nucleocapsid proteins onto the viral genomic RNA in the cytoplasm and budding through the cell membrane embedded with viral encoded glycoproteins. Entry of virus into cells is by endocytosis through clatharin coated pits, fusion of the viral membrane with the endosome, release of the nucleocapsid, and uncoating of the viral genome. During viral replication the genomic 49S RNA serves as template for synthesis of the complementary negative strand. This negative strand in turn serves as template for genomic RNA and an internally initiated 26S subgenomic RNA. The Sindbis viral nonstructural proteins are translated from the genomic RNA while structural proteins are translated from the subgenomic 26S RNA. All viral genes are expressed as a polyprotein and processed into individual proteins by post translational proteolytic cleavage. The packaging sequence resides within the nonstructural coding region, therefore only the genomic 49S RNA is packaged into virions.

Several different Sindbis vector systems may be constructed and utilized within the present invention. Representative examples of such systems include those described within U.S. Pat. Nos. 5,091,309 and 5,217,879, and PCT Publication No. WO 95/07994.

Other Viral Gene Delivery Vectors

In addition to retroviral vectors and alphavirus vectors, numerous other viral vectors systems may also be utilized as a gene delivery vector. Representative examples of such gene delivery vectors include viruses such as pox viruses, such as canary pox virus or vaccinia virus (Fisher-Hoch et al., PNAS 56:317-321, 1989; Flexner et al., Ann. N. Y Acad. Sci. 5(59:86-103, 1989; Flexner et al., Vaccine 5:17-21, 1990; U.S. Pat. Nos. 4,603,112, 4,769,330 and 5,017,487; WO 89/01973); SV40 (Mulligan et al., Nature 277:108-114, 1979); influenza virus (Luytjes et al., Cell 59:1107-1113, 1989; McMicheal et al., N. Eng. J. Med. 509:13-17,1983; and Yap et al., Nature 273:238-239, 1978); herpes (Kit, Adv. Exp. Med. Biol. 275:219-236, 1989; U.S. Pat. No. 5,288,641); HIV (Poznansky, J. Virol (65:532-536, 1991); measles (EP 0 440,219); Semliki Forest Virus, and coronavirus, as well as other viral systems (e.g., EP 0,440,219; WO 92/06693; U.S. Pat. No. 5,166,057). In addition, viral carriers may be homologous, non-pathogenic(defective), replication competent virus (e.g., Overbaugh et al., Science 239:906-910,1988), and nevertheless induce cellular immune responses, including CTL.

Non-Viral Gene Delivery Vectors

In addition to the above viral-based vectors, numerous non-viral gene delivery vectors may likewise be utilized within the context of the present invention. Representative examples of such gene delivery vectors include direct delivery of nucleic acid expression vectors, naked DNA (e.g., DNA not contained in a viral vector) (WO 90/11092), polycation condensed DNA linked or unlinked to killed adenovirus (Curiel et al., Hum. Gene Ther. 3:147-154, 1992), DNA ligand linked to a ligand with or without one of the high affinity pairs described above (Wu et al., J. of Biol. Chem (264:16985-16987, 1989), nucleic acid containing liposomes (e.g., WO 95/24929 and WO 95/12387) and certain eukaryotic cells.

Promoters

Any of a variety of promoters can be used in the gene delivery vectors of the invention to provide for a suitable level or pattern of expression of GDNF. The promoters are generally eukaryotic promoter, i.e., promoters that facilitated expression of an operably linked coding sequence in a eukaryotic cell. Promoters suitable for use in the present invention include constitutive promoters, strong promoters (e.g., CMV promoters), inducible promoters, and tissue-specific or cell-specific promoters (e.g., promoters that preferentially facilitate expression in a limited number of tissues or cell types (e.g., eye tissues, retina, retinal cells, photoreceptor cells, and the like).

In a preferred embodiment, the promoter comprises (from 5′ to 3′) a viral enhancer (preferably a CMV immediate early enhancer), and a beta-actin promoter (preferably Chicken beta-actin promoter-exon 1-intron 1 element). In a specific preferred embodiment, the promoter comprises (from 5′ to 3′) CMV immediate early enhancer (381 bp)/Chicken beta-actin (CBA) promoter-exon 1-intron 1 (1352 bp) element, which together are termed herein the “CBA promoter.”

Conditions Amenable to Treatment

The methods of the present invention are able not only to protect and prevent photoreceptors from degeneration, but also to promote regeneration of retinal cells. The methods of the invention can be used to treat (e.g., prior to or after the onset of symptoms) in a susceptible subject or subject diagnosed with a variety of eye diseases. The eye disease may be a results of environmental (e.g., chemical insult, thermal insult, and the like), mechanical insult (e.g., injury due to accident or surgery), or genetic factors. The subject having the condition may have one or both eyes affected, and therapy may be administered according to the invention to the affected eye or to an eye at risk of photoreceptor degeneration due to the presence of such a condition in the subject's other, affected eye.

The present invention provides methods which generally comprise the step of intraocularly administering (e.g., by subretinal injection) a gene delivery vector which directs the expression of a neurotrophic factor GDNF to the eye to treat, prevent, or inhibit the progression of an eye disease. As utilized herein, it should be understood that the terms “treated, prevented, or, inhibited” refers to the alteration of a disease onset, course, or progress in a statistically significant manner.

Another condition amenable to treatment according to the invention is Age-related Macular Degeneration (AMD). The macula is a structure near the center of the retina that contains the fovea. This specialized portion of the retina is responsible for the high-resolution vision that permits activities such as reading. The loss of central vision in AMD is devastating. Degenerative changes to the macula (maculopathy) can occur at almost any time in life but are much more prevalent with advancing age. Conventional treatments are short-lived, due to recurrent choroidal neovascularization. AMD has two primary pathologic processes, choroidal neovascularization (CNV) and macular photoreceptor cell death. Delivery of GDNF to the eye according to the present invention can ameliorate the photoreceptor cell death. Administration of GDNF has a distinct advantage relative to other NTFs (such as FGF-2) in that GDNF is not angiogenic. Thus GDNF may be the NTF of choice to treat AMD to preserve macular cones without exacerbating the CNV.

Exemplary conditions of particular interest which are amenable to treatment according to the methods of the invention include, but are not necessarily limited to, retinitis pigmentosa (RP), diabetic retinopathy, and glaucoma, including open-angle glaucoma (e.g., primary open-angle glaucoma), angle-closure glaucoma, and secondary glaucomas (e.g., pigmentary glaucoma, pseudoexfoliative glaucoma, and glaucomas resulting from trauma and inflammatory diseases).

Further exemplary conditions amenable to treatment according to the invention include, but are not necessarily limited to, retinal detachment, age-related or other maculopathies, photic retinopathies, surgery-induced retinopathies, toxic retinopathies, retinopathy of prematurity, retinopathies due to trauma or penetrating lesions of the eye, inherited retinal degenerations, surgery-induced retinopathies, toxic retinopathies, retinopathies due to trauma or penetrating lesions of the eye.

Specific exemplary inherited conditions of interest for treatment according to the invention include, but are not necessarily limited to, Bardet-Biedl syndrome (autosomal recessive); Congenital amaurosis (autosomal recessive); Cone or cone-rod dystrophy (autosomal dominant and X-linked forms); Congenital stationary night blindness (autosomal dominant, autosomal recessive and X-linked forms); Macular degeneration (autosomal dominant and autosomal recessive forms); Optic atrophy, autosomal dominant and X-linked forms); Retinitis pigmentosa (autosomal dominant, autosomal recessive and X-linked forms); Syndromic or systemic retinopathy (autosomal dominant, autosomal recessive and X-linked forms); and Usher syndrome (autosomal recessive).

Assessment of Therapy

The effects of therapy according to the invention as described herein can be assessed in a variety of ways, using methods known in the art. For example, the subject's vision can be tested according to conventional methods. Such conventional methods include, but are not necessarily limited to, electroretinogram (ERG), focal ERG, tests for visual fields, tests for visual acuity, ocular coherence tomography (OCT), Fundus photography, Visual Evoked Potentials (VEP) and Pupillometry. In general, the invention provides for maintenance of a subject's vision (e.g., prevention or inhibition of vision loss of further vision loss due to photoreceptor degeneration), slows progression of vision loss, or in some embodiments, provides for improved vision relative to the subject's vision prior to therapy.

Methods of Administration

The gene delivery vectors of the present invention can be delivered to the eye through a variety of routes. They may be delivered intraocularly, by topical application to the eye or by intraocular injection into, for example the vitreous or subretinal (interphotoreceptor) space. Alternatively, they may be delivered locally by insertion or injection into the tissue surrounding the eye. They may be delivered systemically through an oral route or by subcutaneous, intravenous or intramuscular injection. Alternatively, they may be delivered by means of a catheter or by means of an implant, wherein such an implant is made of a porous, non-porous or gelatinous material, including membranes such as silastic membranes or fibers, biodegradable polymers, or proteinaceous material. The gene delivery vector can be administered prior to the onset of the condition, to prevent its occurrence, for example, during surgery on the eye, or immediately after the onset of the pathological condition or during the occurrence of an acute or protracted condition.

The gene delivery vector can be modified to enhance penetration of the blood-retinal barrier. Such modifications may include increasing the lipophilicity of the pharmaceutical formulation in which the gene delivery vector is provided.

The gene delivery vector can be delivered alone or in combination, and may be delivered along with a pharmaceutically acceptable vehicle. Ideally, such a vehicle would enhance the stability and/or delivery properties. The invention also provides for pharmaceutical compositions containing the active factor or fragment or derivative thereof, which can be administered using a suitable vehicle such as liposomes, microparticles or microcapsules. In various embodiments of the invention, it may be useful to use such compositions to achieve sustained release of the active component.

The amount of gene delivery vector (e.g., the number of viral particles), and the amount of GDNF expressed, effective in the treatment of a particular disorder or condition will depend of the nature of the disorder or condition and a variety of patient-specific factors, and can be determined by standard clinical techniques.

In a preferred embodiment, the gene delivery vectors are administered to the eye, preferably intraocularly to a variety of locations within the eye depending on the type of disease to be treated, prevented, or, inhibited, and the extent of disease. Examples of suitable locations include the retina (e.g., for retinal diseases), the vitreous, or other locations in or adjacent the retina or in or adjacent the eye.

Briefly, the human retina is organized in a fairly exact mosaic. In the fovea, the mosaic is a hexagonal packing of cones. Outside the fovea, the rods break up the close hexagonal packing of the cones but still allow an organized architecture with cones rather evenly spaced surrounded by rings of rods. Thus in terms of densities of the different photoreceptor populations in the human retina, it is clear that the cone density is highest in the foveal pit and falls rapidly outside the fovea to a fairly even density into the peripheral retina (see Osterberg, G. (1935) Topography of the layer of rods and cones in the human retina. Acta Ophthal. (suppl.) 6, 1-103; see also Curcio, C. A., Sloan, K. R., Packer, O., Hendrickson, A. E. and Kalina, R. E. (1987) Distribution of cones in human and monkey retina: individual variability and radial asymmetry. Science 236, 579-582).

Access to desired portions of the retina, or to other parts of the eye may be readily accomplished by one of skill in the art (see, generally Medical and Surgical Retina: Advances, Controversies, and Management, Hilel Lewis, Stephen J. Ryan, Eds., medical—” illustrator, Timothy C. Hengst. St. Louis: Mosby, c1994. xix, 534; see also Retina, Stephen J. Ryan, editor in chief, 2nd ed., St. Louis, Mo.: Mosby, c1994. 3 v. (xxix, 2559 p.).

The amount of the specific viral vector applied to the retina is uniformly quite small as the eye is a relatively contained structure and the agent is injected directly into it. The amount of vector that needs to be injected is determined by the intraocular location of the chosen cells targeted for treatment. The cell type to be transduced will be determined by the particular disease entity that is to be treated.

For example, a single 20-microliter volume (e.g., containing about 10¹³ physical particle titer/ml rAAV) may be used in a subretinal injection to treat the macula and fovea of a human eye. A larger injection of 50 to 100 microliters may be used to deliver the rAAV to a substantial fraction of the retinal area, perhaps to the entire retina depending upon the extent of lateral spread of the particles.

A 100 microliter injection will provide several million active rAAV particles into the subretinal space. This calculation is based upon a titer of 10¹³ physical particles per milliliter. Of this titer, it is estimated that 1/1000 to 1/10,000 of the AAV particles are infectious. The retinal anatomy constrains the injection volume possible in the subretinal space (SRS). Assuming an injection maximum of 100 microliters, this would provide an infectious titer of 10^{s} to 10⁹ rAAV in the SRS. This would have the potential of infecting all of the approximately 150×10⁶ photoreceptors in the entire human retina with a single injection.

Smaller injection volumes focally applied to the fovea or macula may adequately transfect the entire region affected by the disease in the case of macular degeneration or other regional retinopathies.

Gene delivery vectors can alternately be delivered to the eye by intraocular injection into the vitreous, e.g., to treat glaucomatous loss of retinal ganglion cells through apoptosis. In this application, the primary target cells to be transduced are the retinal ganglion cells, which are the retinal cells primarily affected in glaucoma. In this application, the injection volume of the gene delivery vector could be substantially larger, as the volume is not constrained by the anatomy of the subretinal space. Acceptable dosages in this instance can range from 25 microliters to 1000 microliters.

Pharmaceutical Compositions

Gene delivery vectors can be prepared as a pharmaceutically acceptable composition suitable for administration. In general, such pharmaceutical compositions comprise an amount of a gene delivery vector suitable for delivery of GDNF-encoding polynucleotide to a cell of the eye for expression of a therapeutically effective amount of GDNF, combined with a pharmaceutically acceptable carrier or excipient. Preferably, the pharmaceutically acceptable carrier is suitable for intraocular administration. Exemplary pharmaceutically acceptable carriers include, but are not necessarily limited to, saline or a buffered saline solution (e.g., phosphate-buffered saline).

Various pharmaceutically acceptable excipients are well known in the art. As used herein, “pharmaceutically acceptable excipient” includes any material which, when combined with an active ingredient of a composition, allows the ingredient to retain biological activity, preferably without causing disruptive reactions with the subject's immune system or adversely affecting the tissues surrounding the site of administration (e.g., within the eye).

Exemplary pharmaceutically carriers include sterile aqueous of non-aqueous solutions, suspensions, and emulsions. Examples include, but are not limited to, any of the standard pharmaceutical excipients such as a saline, buffered saline (e.g., phosphate buffered saline), water, emulsions such as oil/water emulsion, and various types of wetting agents. Examples of non-aqueous solvents are propylene glycol, polyethylene glycol, hyaluronic acid, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles can include fluid and nutrient replenishers, electrolyte replenishers (such as those based on Ringer's dextrose), and the like.

A composition of gene delivery vector of the invention may also be lyophilized using means well known in the art, for subsequent reconstitution and use according to the invention. Where the vector is to be delivered without being encapsulated in a viral particle (e.g., as “naked” polynucleotide), formulations for liposomal delivery, and formulations comprising microencapsulated polynucleotides, may also be of interest.

Compositions comprising excipients are formulated by well known conventional methods (see, for example, Remington's Pharmaceutical Sciences, Chapter 43, 14th Ed., Mack Publishing Col, Easton Pa. 18042, USA).

In general, the pharmaceutical compositions can be prepared in various forms, preferably a form compatible with intraocular administration. Stabilizing agents, wetting and emulsifying agents, salts for varying the osmotic pressure or buffers for securing an adequate pH value may also optionally be present in the pharmaceutical composition.

The amount of gene delivery vector in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The pharmaceutical composition can comprise other agents suitable for administration, which agents may have similar to additional pharmacological activities to the therapeutic protein to be delivered (e.g., GDNF).

Kits

The invention also provides kits comprising various materials for carrying out the methods of the invention. In one embodiment, the kit comprises a vector encoding a GDNF polypeptide, which vector is adapted for delivery to a subject, particularly an eye of the subject, and adapted to provide for expression of the GDNF polypeptide in a cell of an eye, particularly a mammalian cell. The kit can comprise the vector in a sterile vial, which may be labeled for use. The vector can be provided in a pharmaceutical composition. In one embodiment, the vector is packaged in a virus. The kit can further comprise a needle and/or syringe suitable for use with the vial or, alternatively, containing the vector, which needle and/or syringe are preferably sterile. In another embodiment, the kit comprises a catheter suitable for delivery of a vector to the eye, which catheter may be optionally attached to a syringe for delivery of the vector. The kits can further comprise instructions for use, e.g., instructions regarding route of administration, dose, dosage regimen, site of administration, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Materials and Methods

The following materials and methods were used in the Examples that follow.

Animals

The examples below use a transgenic rat model (TgN S334ter-4) expressing a mutated rhodopsin gene in which a termination codon is present at residue 334 of the opsin transgene resulting in a protein lacking the 15 C-terminal amino acids. The C-terminus is involved in rhodopsin localization to the outer segments and its absence contributes to photoreceptor cell death by a caspase-3 dependent mechanism (Liu, et al. (1999) J. Neurosci. 19: (12), 4778-4785; Green, et al. (2000) Invest. Ophthalmol. Vis. Sci. 41: (6), 1546-1553). Multiple mutations within the C-terminus have been identified in patients with RP. Thus, using TgN S334ter-4 rats enables us to design and test therapies in an animal model with a disease similar to human RP. The retinas of heterozygous TgN S334ter-4 rats develop normally and have 8-10 rows of photoreceptor nuclei in the outer nuclear layer (ONL) at post-natal day 15 (P15). The time course of degeneration occurs in two phases beginning at about P15. The first phase between P15 and P60 is fast with the ONL degenerating to 2-3 rows of nuclei accompanied by a substantially reduced eletroretinographic response by P60. Beyond P60 a slower rate of ONL loss ensues.

The transgenic S334ter-4 rats were produced on a Sprague-Dawley background (Chrysalis DNX Transgenic Sciences, Princeton, N.J.). Our research lab cared for rats in accordance with the guidelines of the University of California, Berkeley Committee on Animal Research. Rats heterozygous for the S334ter-4 transgene (Simonsen, Gilroy, Calif.) in experiments discussed. Animals were raised on a 12 hr light/12 hr dark schedule at in-cage illuminance of approximately 15 foot-candles.

rAAV Vector Expressing GDNF

The recombinant AAV vectors were based on pTR-UF construct (Flannery, et al. (1997) Proc Natl. Acad. Sci. USA 94: (13), 6916-6921) in which the opsin promoter was replaced with a CMV immediate early enhancer (381 bp)/Chicken beta-actin (CBA) promoter-exon 1-intron 1 (1352 bp) element which together are termed the CBA promoter. This is followed by a poliovirus internal ribosome entry sequence (IRES, 637 bp). Together this sequence supports expression in photoreceptors, RPE, and ganglion cells (Li and Hauswirth, unpublished data, 2000). To create the two vectors used in this study, either green fluorescent protein (GFP) or GDNF cDNA was placed downstream of the IRES element via flanking NotI sites and the orientation and reading frame confirmed by DNA sequence analysis. Plasmid DNA containing this construct this construct into AAV particles employing iodixanol gradient purification followed by heparin-sepharose agarose column chromatography (Hauswirth, et al. (2000) Methods Enzymol. 316: (10), 743-761). AAV-CBA-GDNF vector titer was 9.9×10¹⁰ particles/ml. AAV-CBA-GFP vector titer was 2×10¹² particles/ml. Contaminating helper adenovirus and wild-type AAV titers, assayed by serial dilution cytopathic effect or infectious center assay respectively, were lower than our detection limit of six orders of magnitude below the vector AAV titer.

Subretinal Injections

At P15, rats were anesthetized by an intramuscular injection of ketaminexylazine and their eyes dilated using 2.5% phenylephrine hydrochloride and 1% atropine sulfate. All subretinal infections were performed using a stereomicroscope. A 3 μl volume of virus was injected through a blunt 32 gauge Hamilton syringe by a medial approach. The tip of the needle was inserted through the nasal sclera, choroid, retina, and vitreous, and then the needle repenetrated the superior central retina to deliver the inoculum into the subretinal space. This approach was most successful in avoiding damage to the lens. A total of 3×10⁸ viral particles of either AAV-CBA-GDNF or AAV-CBA-GFP was injected. Introduction of larger volumes to the subretinal space created injury-related effects, such as a rosette formation of photoreceptor cell layers occurring during re-attachment. Delivery of 3 μl was the largest volume that could safely be delivered without injury. TgN S334ter-4 animals were subretinally injected with either AAV-CBA-GDNF (n=44 eyes) or AAV-CBA-GFP (n=29 eyes). Different subgroups of animals were utilized for western blot analysis, immunohistochemistry, RT-PCR, morphometry, and ERG experiments.

Western Blot Analysis

Individual retinas were homogenized by sonication in 5 mM tris-acetate buffer with 65 mM NaCl, 2 mM MgCl₂, and protease inhibitors at 5 weeks post-treatment. Samples were deglycosylated samples with N-glycosidase F. Retinal protein from an AAV-CBA-GDNF-treated animal, an AAV-CBA-GFP-treated animal, human recombinant GDNF protein (R&D Systems, Minneapolis, Minn.), or kaleidoscope pre-stained maker (Bio-rad, Hercules, Calif.) was electrophoresed on a 14% SDS polyacrylamide gel under reducing conditions and transferred to PVDF membrane (Bio-rad, Hercules, Calif.). The blotted membrane was then blocked and incubated with human GDNF antibody at a 1:1000 dilution (Sigma RBI, St. Louis, Mo.), washed and then incubated with horseradish peroxidase-conjugated secondary antibody (Sigma, St. Louis, Mo.). Label was detected with a Renaissance enhanced chemiluminescence system (NEN Life Science Products, Boston, Mass.) and hyperfilm-ECL X-ray film (Amersham Life Science Inc, Arlington Heights, Ill.).

Immunohistochemistry

Rat eyes were enucleated from animals injected with AAV-CBA-GDNF (n=10 eyes) or AAV-CBA-GFP (n=10 eyes) at 5 weeks post-injection, P50. Eyecups were fixed in 4% formaldehyde in PBS for 1 h at room temperature and washed in PBS three times. Eyes were cryoprotected in 30% sucrose overnight at 4° C. and embedded in OCT for at least 2 h at 0° C. Sections were cut 25 μM thick using a CM1850 cryostat (Leica, Nussloch, Germany) and allowed to dry overnight. Sections were incubated for 2 h at room temperature using an antibody to human GDNF (Sigma RBI, St. Louis, Mo.), diluted 1:500 in 1% fetal calf serum, 1% bovine serum albumin, and 0.3% Triton X-100 in PBS. Bound antibodies were detected by incubating sections for 1 h at room temperature with rabbit anti-goat IgG antibodies conjugated to Cy3 (Sigma, St. Louis, Mo.), and/or rabbit anti-goat IgG antibodies conjugated to FITC (Sigma, St. Louis, Mo.). Images were acquired using Zeiss Axiophot fluorescent microscope (Thornwood, N.Y.).

RT-PCR Analysis of GDNF mRNA

Total RNA was isolated from individual eyes injected with AAV-CBA-GDNF (n=5 eyes) and AAV-CBA-GFP-injected (n=5 eyes) retinas at 5 weeks post-injection with standard protocol (Qiagen RNeasy Kit Qiagen, Valencia, Calif.). cDNAs were synthesized using Clontech Advantage RT-for-PCR Kit (Clontech, Palo Alto, Calif.). Kits were used as instructed by manufacturer. cDNAs from individual eyes were subjected to PCR amplification using the following primers. The upstream primer, 5′-ATGAAGTTATGGGATGTCGT-3′, and the downstream primer, 5′-CAGGGTCAGATACATCCACA-3′, amplified both rat and human GDNF cDNA producing a 641 bp band. A second downstream primer, 5′-TCACCAGCCTTCTATTTCTG-3′, was designed that specifically amplified only the human GDNF gene (Frasson, et al. (1999) Invest. Ophthalmol. Vis. Sci. 40: (11), 27242734). When this human GDNF specific primer was used in combination with the upstream primer above, it produced a 511 bp band. Amplified products were visualized on a 1.5% agarose gel stained with ethidium bromide.

Microscopy and Morphological Analysis

Rats were sacrificed at P60, 45 days post-injection by carbon dioxide asphyxiation and cardiac perfusion using 2.5% glutaraldehyde and 2% formaldehyde in PBS. For light microscopy, eyecups were embedded in epon-araldite resin and 1 μm thick sections cut along the vertical meridian in the same plane as the optic nerve. Tissue sections were aligned such that rod outer segments were continuous throughout the plane. Twenty-seven separate measurements of the outer nuclear layer (ONL) thickness were made around the inferior or superior regions using Bioquant 98 image analysis system (R&M Biometrics, Nashville, Tenn.) (LaVail, et al. (1987) Invest Ophthalmol. Vis. Sci. 28: (7), 1043-1048). These measurements from either the superior or inferior regions were averaged to obtain the mean ONL thickness. Statistical significance of the differences in mean ONL thickness between groups was evaluated by Mann-Whitney tests. Morphometric analyses were performed on AAV-CBA-GDNF-injected eyes (n=24), AAV-CBA-GFP-injected eyes (n=9), and uninjected eyes (n=9).

Electroretinography

TgN S334ter-4 rats were treated with either AAV-CBA-GDNF (n=9 eyes), AAV-CBA-GFP (n=9 eyes), or untreated (n=9 eyes). Forty-five days after treatment, P60, rats were dark adapted overnight and anesthetized with an intramuscular injection of xylazine and ketamine. A drop of 0.5% proparacaine hydrochloride was applied to the cornea and pupils were dilated with 2.5% phenylephrine. Contact lenses containing electrodes were placed on the cornea, and reference electrodes were placed subcutaneously under each eye. Stimuli were presented at intensities of −3.886, −1.896, and 0.173 log candela-seconds (cd-s)/m² at 15-s, 30-s, and 1-min intervals. 10 μsec flashes of white light elicited full-field scotopic ERGs, and responses were recorded using a UTAS-E 2000 Visual Electrodiagnostic System (LKC Technologies). The a-wave amplitudes were measured from the baseline to the peak in the corneal negative direction, and b-wave amplitudes from the corneal negative peak to the major corneal positive peak after subtracting any contributions due to oscillatory potentials. Four responses at each intensity were averaged. Statistical significance of amplitude differences was determined by Student's t-test.

### Example 1

### Expression of the GDNF Transgene

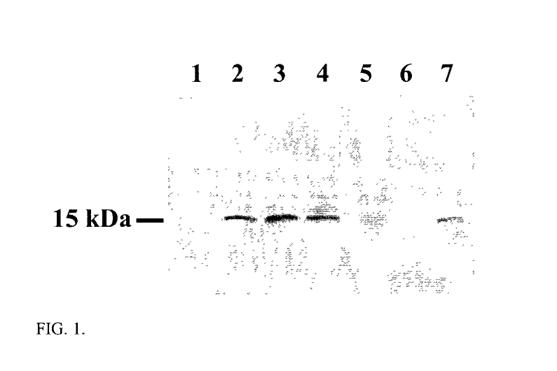

Western blotting was used to analyze expression of human recombinant GDNF in retinas at 5 weeks post-treatment, P50. Human recombinant GDNF was detected in retinas transduced with AAV-CBA-GDNF, but not in untransduced or AAV-CBA-GFP-transduced retinas (FIG. 1). The molecular weight of the GDNF protein produced in AAV-CBA-GDNF-treated animals was approximately 15 kDa, comparable to the unglycosylated and reduced form of human recombinant GDNF (Lin, et al. (1993) Science 260: (5111), 1130-1132).

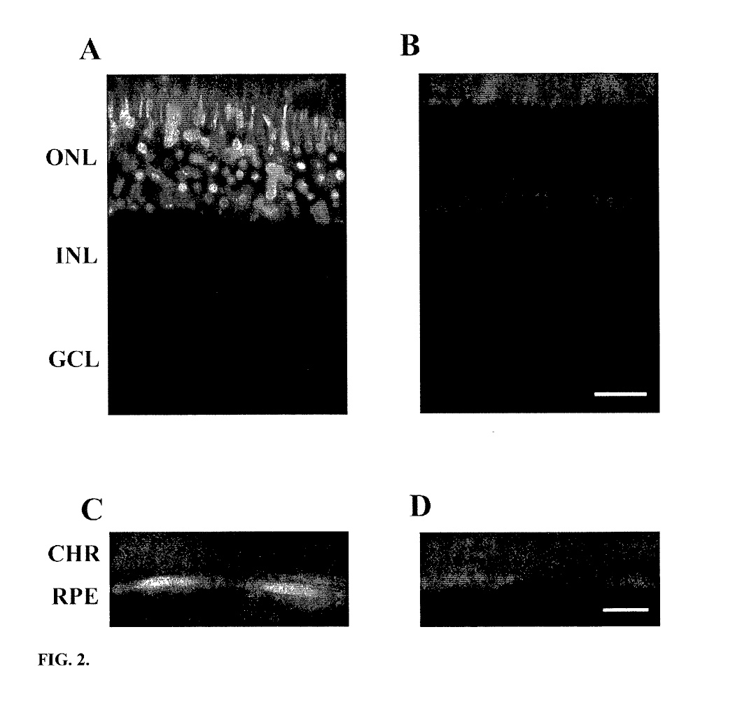

The cell types expressing AAV-CBA-GDNF-derived human recombinant GDNF protein were localized by immunohistochemistry at 5 weeks post-treatment, P50 (FIG. 2). In cryosections of retinas, we observed GDNF in photoreceptor nuclei, inner and outer segments (FIG. 2, Panel A), and in retinal pigment epithelium (RPE) (FIG. 2, Panel C). The GDNF expression level was equally robust in both photoreceptors and RPE. Results for FIG. 2A are representative for the treated area and we did not see labeling in any other retinal cell types. Staining for human recombinant GDNF was absent in uninjected retinas (FIG. 2, Panel B and Panel D). Ten out of ten eyes injected with AAV-CBA-GDNF stained positive for GDNF. The level of expression was uniform across the injection site and appeared consistent in all ten eyes examined.

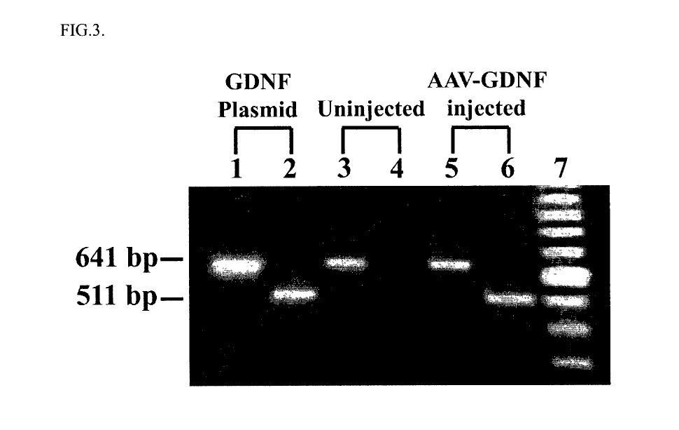

To further document the effect of AAV-CBA-GDNF transduction, samples were assayed for production of GDNF mRNA. Human GDNF transcripts were present in AAV-CBA-GDNF-treated neural retinas, as demonstrated by RT-PCR analysis at 5 weeks post-injection, P50 (FIG. 3, lane 6). In contrast, only endogenous rat GDNF mRNA transcripts were detected in an untreated rat retina (lane 3). Taken together, Western blot, immunohistochemical, and RT-PCR results indicate that AAV-CBA-GDNF can transduce photoreceptors, and the CBA promoter drives expression of human recombinant GDNF in these cells.

### Example 2

### Morphological Analysis of Photoreceptor Rescue

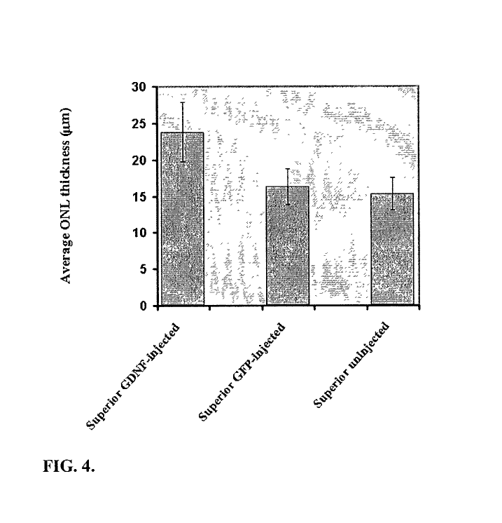

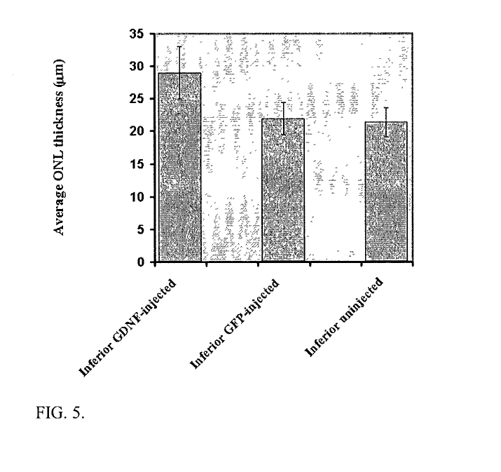

TgN S334ter-4 animals were injected with either AAV-CBA-GDNF or AAV-CBA-GFP at P15 and assessed the morphological consequences of expressing GDNF in the degenerating rat retina on P60, 45 days post-injection. The amount of photoreceptor degeneration by examined by measuring ONL thickness, since thinning of the ONL occurs as photoreceptors die by apoptosis. Because ONL thickness varies between the inferior and superior regions of the eye in TgN S334ter-4 animals (Lau et al. (2000) Invest. Ophthalmol. Vis. Sci. 41(11):3622-3633), data is presented from each hemisphere separately. In both the superior and inferior regions of AAV-CBA-GDNF injected eyes, there was a significant increase in ONL thickness in comparison to control-affected eyes. In the superior hemisphere, eyes injected with AAV-CBA-GDNF an had ONL thickness of 23.8±4.1 μm as compared to controls injected with AAV-CBA-GFP (16.3±2.5 μm) or uninjected eyes (15.4±2.2 μm) (FIG. 4). In the inferior hemisphere, eyes injected with AAV-CBA-GDNF had an ONL thickness of 28.8±2.8 μm as compared to controls injected with AAV-CBA-GFP (21.9±3.0 μm) or uninjected eyes (21.4±2.6 μm) (FIG. 5).

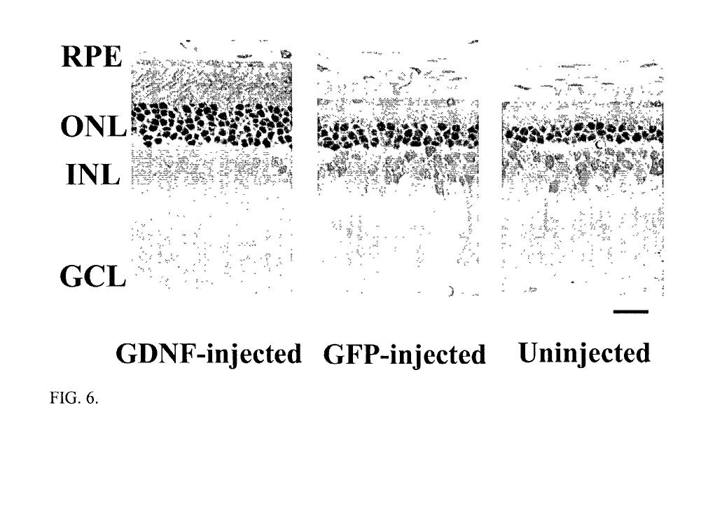

Statistical analysis indicates that ONL thickness measurements, in both the superior and inferior regions of AAV-CBA-GDNF-injected retinas, were significantly increased in comparison to AAV-CBA-GFP-injected, or uninjected controls (P<0.0002). Although the ONL thickness of AAV-CBA-GFP treated controls was slightly thicker than uninjected controls, they were not significantly different (P>0.05) in either the superior or inferior regions. In the superior region, retinas injected with AAV-CBA-GDNF had an ONL composed of 6 to 7 rows of photoreceptor nuclei, compared to 3 to 4 rows in AAV-CBA-GFP, and 2 to 3 rows in uninjected controls at P60 (FIG. 6). In addition to increased ONL thickness, AAV-CBA-GDNF treated retinas generally had rod inner and outer segments that were continuous and well organized. In contrast, AAV-CBA-GFP treated or untreated affected controls display disorganized photoreceptor inner and outer segments. In summary, retinas injected with AAV-CBA-GDNF showed significant morphological rescue by both increased ONL thickness and better preserved photoreceptor inner and outer segments.

### Example 3

### Physiological Analysis of Photoreceptor Rescue

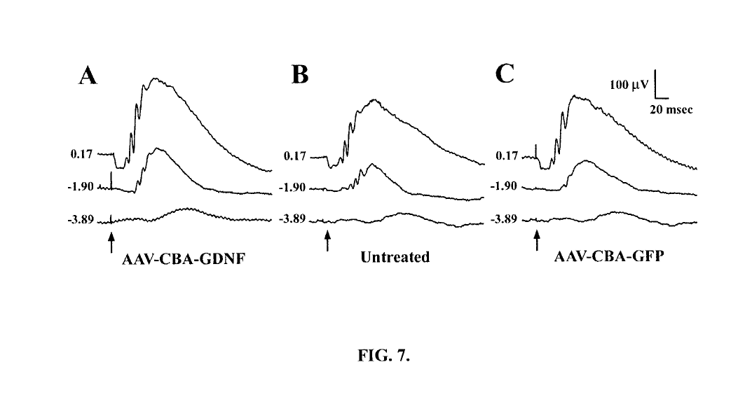

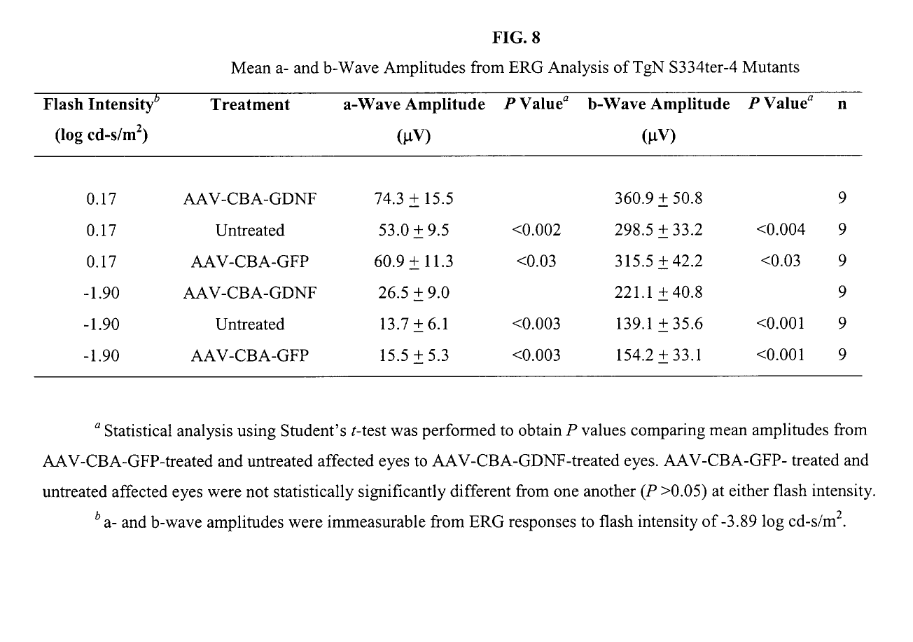

TgN S334ter-4 animals were also assessed for physiological rescue by analysis of the scotopic electroretinogram (ERG) at P60. FIG. 7 shows representative tracings of scotopic (rod-mediated) responses from TgN S334ter-4 animals treated with AAV-CBA-GDNF, AAV-CBA-GFP, or untreated. This functional analysis of treated eyes reflected the protection from cell death observed by morphological analysis. Control-affected eyes exhibited rod-mediated ERGs with severely impaired responses. Examination of ERGs from eyes injected with AAV-CBA-GDNF showed increased physiological functioning in comparison to eyes injected with AAV-CBA-GFP or uninjected control-affected eyes. Mean a- and b-wave amplitudes of the scotopic ERG response were measured at the two highest stimulus intensities (FIG. 8). Statistical analysis indicated that mean a- and b-wave amplitudes of AAV-CBA-GDNF-injected retinas were significantly increased compared to AAV-CBA-GFP-injected or uninjected control-affected retinas at both flash intensities. The mean a- and b-wave amplitudes of either AAV-CBA-GFP-treated or uninjected control-affected retinas were not significantly different from one another at either intensity.

### Example 4

### Absence of Pathology after rAAV Inoculation

Histological sections of AAV-CBA-GDNF-injected retinas were examined visually for signs of inflammation, mitogenesis, neovascularization, and other pathological effects of viral transduction or neurotrophic factor expression. Photoreceptor rosette formations were observed, which result from slight misalignment during retinal reattachment, in equal numbers in both experimental and control-injected eyes. There were no other structural abnormalities and no indications that nerve or glial cell mitogenesis had been induced by the GDNF treatment. Injected eyes also showed no increases in macrophage populations that would indicate an inflammatory response, and no signs of neovascularization in either the choroidal or retinal vasculatures. Thus, AAV transduction and growth factor expression did not produce any detectable pathology in the retina.

Discussion of Examples

Expression of GDNF reproducibly slowed photoreceptor cell death in TgN S334ter-4 rat retinas for at least 45 days. Expression, as documented by Western blot, immunohistochemistry, and RT-PCR analysis, indicated that mRNA was produced and human recombinant GDNF was expressed in the rat retina. The results above show that the CBA promoter led to a high level of vector-derived protein expression and we localized uniform GDNF labeling to photoreceptors and RPE cells. Labeling was not detected for endogenous rat GDNF protein, suggesting that it is not normally expressed at high enough concentrations in the retina to reach detection levels for immunohistochemistry. However, a low level of endogenous GDNF is present in the rat retina because rat specific GDNF mRNA transcripts were detected by RT-PCR. Based on the results of an earlier study in which AAV with the CBA promoter demonstrated transduction of RPE cells in-vivo in the retina (Acland, et al. (2001) Nature Genetics 28: (1), 92-95), labeling of GDNF seen in the immunohistochemical study here likely results from transduction of RPE cells. However, it is possible that the RPE phagocytose vector derived GDNF during photoreceptor disc shedding or that photoreceptors through paracrine secretion provide the RPE with GDNF.

Vector derived GDNF expression resulted in significant morphological and physiological GDNF rescue of photoreceptor degeneration in TgN S334ter-4 rat. Mean superior ONL thickness from AAV-CBA-GDNF-treated retinas was on average 47% greater than in AAV-CBA-GFP-treated retinas (23.8±4.1 μm vs. 16.3±2.5 μm), indicating a greater number of surviving rod photoreceptors. AAV-CBA-GDNF-treated treated retinas had 22% larger average a-wave mean amplitudes using the 0.17 log cd-s/m² stimulus intensity compared to AAV-CBA-GFP-treated controls (74.3±15.5 μV vs. 60.9±11.3 μV), indicating an overall improvement in photoreceptor functioning. Thus, the degree of morphological rescue and photoreceptor inner and outer segment structural preservation correlated well with increases in the physiological response of photoreceptors. Because w the same AAV vector driving the GFP reporter gene was used as a control, we conclude that morphological and functional rescue seen in AAV-CBA-GDNF-treated retinas arises directly from the survival-enhancing properties of GDNF.

While an understanding of the mechanism through which GDNF works to effect protection of photoreceptors is not required to work the invention, a discussion of the possible mechanisms is instructive. Although it has not yet been determined which cell types respond to GDNF and promote photoreceptor survival, clearly photoreceptors must respond either directly or indirectly through an intermediate retinal cell type. Retinal cells which lack the GFRA1 receptor may remain capable of responding to GDNF because GFRA1 may be soluble (Jing, et al. (1996) Cell 85: (7), 1113-1124). Alternatively, it is possible that the observed photoreceptor survival could be due to the effects of the neurotrophic factor on Müller cells or other retinal cells. The soluble complex of GDNF and GFRA1 may bind to any glial or neuronal cell that expresses the membrane-bound tyrosine kinase receptor, RET (Takahashi, et al. (1987) Mol Cell Biol 7: (4), 1378-1385). Furthermore, it has been demonstrated that intraocular injections of GDNF proteins have led to upregulation of glial fibrillary acidic protein expression, suggesting that GDNF may regulate phenotypic expression of Müller cells and that GDNF's effects on photoreceptor survival may be mediated by Müller cells, via an indirect pathway (Frasson, et al. (1999) Invest. Ophthalmol. Vis. Sci. 40: (11), 2724-2734). Additionally, GDNF has been shown to provide trophic support to sensory neurons (Matheson, et al. (1997) J. Neurobiol. 32: (1), 22-32), and GDNF mRNA is expressed during development in the retina (Nosrat, et al. (1996) Cell Tissue Res. 286: (2), 191-207).

The intracellular mechanism of protection from degeneration and the physiological roles of GDNF over expression in the present experiments are yet to be elucidated. However, different concentrations of a trophic factor ligand available for receptor binding can determine the nature of the molecular response within a target cell (Marshall (1995) Cell 80: (2), 179-185). The second messenger pathway is only transiently activated at low levels of neurotrophic factors, while high concentration can result in novel gene expression. Therefore, it is possible that the elevated level of vector derived GDNF in TgN S334ter-4 animals may ultimately lead to gene products that interfere with photoreceptor apoptotic cell death. Thus, it remains unresolved as to how GDNF might exert its trophic effects; several retinal cell types, including photoreceptors, glial cells, and other retinal neurons remain candidates.

An increase of approximately 47% in ONL thickness was observed in the superior hemisphere due to AAV-CBA-GDNF treatment when compared to AAV-CBA-GFP treatment. Unlike fibroblast growth factors (Gerwins, et al. (2000) Crit. Rev. Oncol. Hematol. 34: (3), 185-194), GDNF is not reported to be angiogenic and thus should not lead to neovascular complications, making it a particularly good candidate for neuroprotection in the eye. Our histologic analysis recorded neither the presence of abnormal numbers of macrophages nor the growth of new blood vessels from either the choroidal or retinal vasculature. It is significant that the AAV-CBA-GDNF treatment, containing viral capsid protein and expressing human GDNF, produced no visible signs of nerve or glial cell mitogenesis or inflammation in any of the eyes studied. This lack of immune response may in part reflect the immune-privileged status of the eye (Wenkel et al. (1998) Invest Ophthalmol. Vis. Sci. 39: (10), 1823-1834).

Because AAV vectors are able to mediate the focal delivery of GDNF at the site of photoreceptor degeneration, AAV-CBA-GDNF treatment is applicable to treatment of humans. Gene therapy approaches that lead to long-term expression appear to avoid the requirement for repeated injections of recombinant protein and avoid side effects of bolus delivery. The results presented here, using a rodent model of human RP, support the conclusion that neuroprotective therapy will retard or prevent the onset of blindness associated with the lifetime course of retinal degenerative diseases in humans.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the invention and within the scope of the claims which claim priority to the present application.

That which is claimed is:

1. A method for treating or preventing photoreceptor degeneration in a subject having or susceptible to an eye disease or condition associated with photoreceptor degeneration, the method comprising:
administering to the subject in need of therapy a recombinant gene delivery vector adapted for expression of a GDNF polypeptide in an eye of the subject;
wherein said administering provides for production of the GDNF polypeptide in an eye of the subject in an amount sufficient to treat or prevent photoreceptor degeneration in the eye of the subject.

2. The method of claim 1, wherein the vector is administered to the eye of the subject.

3. The method of claim 2, wherein said administering is by intraocular administration.

4. The method of claim 2, wherein said administering is by subretinal administration.

5. The method of claim 1, wherein the recombinant gene delivery vector is a viral vector.

6. The method of claim 5, wherein the viral vector is a recombinant adeno-associated viral vector.

7. The method of claim 1, wherein photoreceptor degeneration or risk of photoreceptor degeneration in the subject is associated with retinitis pigmentosa (RP), glaucoma, retinal detachment, age-related maculopathy, photic retinopathy, surgery-induced retinopathy, toxic retinopathy, retinopathy of prematurity, retinopathy as a result of trauma, retinopathy as a result of a penetrating lesion, or an inherited retinal degeneration.

8. A method for treating or preventing photoreceptor degeneration in a subject having or susceptible to photoreceptor degeneration, the method comprising:
administering to an eye in need of therapy in the subject a recombinant gene delivery vector adapted for expression of a GDNF polypeptide in a retinal cell of the eye;
wherein said administering provides for production of the GDNF polypeptide in the retinal cell in an amount sufficient to treat or prevent photoreceptor degeneration in the eye of the subject.

9. The method of claim 8, wherein said administering is by intraocular administration.

10. The method of claim 8, wherein said administering is by subretinal administration.

11. The method of claim 8, wherein the recombinant gene delivery vector is a viral vector.

12. The method of claim 11, wherein the viral vector is a recombinant adeno-associated viral vector.

13. The method of claim 8, wherein photoreceptor degeneration or risk of photoreceptor degeneration in the subject is associated with retinitis pigmentosa (RP), glaucoma, retinal detachment, age-related maculopathy, photic retinopathy, surgery-induced retinopathy, toxic retinopathy, retinopathy of prematurity, retinopathy as a result of trauma, retinopathy as a result of a penetrating lesion, or an inherited retinal degeneration in the subject.

14. A method of treating or preventing photoreceptor degeneration in an eye of a subject having retinitis pigmentosa (RP), the method comprising:
administering to an eye in need of therapy in the subject a recombinant adeno-associated viral vector adapted for expression of a GDNF polypeptide in a retinal cell of the eye;
wherein said administering provides for production of the GDNF polypeptide in the retinal cell in an amount sufficient to treat or prevent photoreceptor degeneration in the eye of the subject having RP.

15. The method of claim 8, wherein said administering is by intraocular administration.

16. The method of claim 8, wherein said administering is by subretinal administration.

17. A kit adapted for use in the method of claim 1, 8 or 14, the kit comprising:
a sterile container containing a recombinant gene delivery vector adapted for expression of a GDNF polypeptide in an eye of the subject.

18. The kit of claim 17, wherein the kit comprises a sterile needle adapted for injection of the recombinant gene delivery vector into an eye of the subject.
